(19) United States
(12) Patent Application Publication
(10) Pub. No.: US 2023/0073367 A1
(43) Pub. Date: Mar. 9, 2023

(54) Systems and Methods for Identifying Food Processing and Prescribing a Diet

(71) Applicant: Northeastern University, Boston, MA (US)

(72) Inventors: Giulia Menichetti, Boston, MA (US); Albert-László Barábasi, Brookline, MA (US)

(21) Appl. No.: 17/760,280

(22) PCT Filed: Feb. 5, 2021
(86) PCT No.: PCT/US2021/016865

§ 371 Date: Aug. 5, 2022

Related U.S. Application Data
(60) Provisional application No. 62/971,128, filed on Feb. 6, 2020.

(51) Int. Cl. G16H 20/60; G06N 20/20; G09B 19/00

(52) U.S. Cl. CPC G16H 20/60; G06N 20/20; G09B 19/0092

(57) ABSTRACT

Systems and methods for identifying a degree of food processing based on food nutrient content are provided. Given in a nutrient profile for a food that includes nutrient content data for the food, a vector of probabilities is generated in which each probability of the vector represents a probability associated with a processing category for the food. A food processing score is determined based on the vector of probabilities and displayed. An individual food score can also be determined based on a plurality of food processing scores. The individual food processing score can be weight-based or calorie-based.

### RELATED APPLICATION

This application claims the benefit of U.S. Provisional Application No. 62/971,128, filed on Feb. 6, 2020. The entire teachings of the above application are incorporated herein by reference.

### BACKGROUND

Unhealthy diet is a major risk factor for multiple noncommunicable diseases, from coronary heart disease (CHD), to cancer and diabetes, together accounting for 70% of mortality and 58% of morbidity worldwide. Distinct from malnutrition and nutrient deficiencies, these diseases are not caused by insufficient nutrient intake or absorption, but are the cumulative effect of dietary choices that span multiple years.

Traditionally, dietary recommendations like the food Pyramid (1992) and MyPlate (2011) have been used to combat the food epidemic, defining an appropriate mix of fruits vegetables, grains, dairy, and protein foods that constitute a healthy diet. In recent years, however, an increasing number of dietary guidelines have shifted their attention to the role of processed food in our diet, prompted by observational studies and meta-analyses showing how dietary patterns such as prudent, healthy, vegetarian, Nordic, and Mediterranean, which rely on unprocessed foods, are more protective than the processing-heavy Western diet against disease onset. Indeed, while humans as hunters-gatherers were exposed to a variety of food sources, from plants to animals, the introduction of novel staple foods fundamentally altered several key nutritional characteristics of ancestral diets, ultimately affecting population health. As foods like refined cereals, refined sugars, refined vegetable oils, fatty meats, and salt gradually displaced the minimally processed diets that rely on wild plants and animal products, the foods adversely affected dietary indicators such as glycemic load, fatty acid composition, macronutrient composition, micro-nutrient density, acid-base balance, sodium-potassium ratio, and fiber content.

The understanding of the health implications of processed and ultra-processed food has benefited from the introduction of the NOVA index, which categorizes individual foods according to the extent and purpose of the processing and focuses on food production rather than food nutrient content. NOVA has enabled multiple epidemiological studies to investigate the association between consumption of ultra-processed food and disease onset, documenting increased risk of CHD, diabetes mellitus, cancer, and depressive symptoms. Despite its success, the widespread use of the NOVA classification system remains limited.

### SUMMARY

Systems and methods are provided for identifying a degree of food processing. The systems and methods provided can identify a degree of food processing based on food nutrient content, such as is available by food composition databases and food composition information provided by food manufacturers. Precision nutrition systems and methods are also provided in which prescriptions can be provided to an individual based, at least in part, on determined food processing scores and, optionally, based on biological data relating to the individual.

A system for identifying a degree of food processing based on food nutrient content includes a data source that includes a nutrient profile for a food and a processor communicatively coupled to the data source. The nutrient profile includes nutrient content data for the food. The processor is configured to generate a vector of probabilities based on the nutrient profile for the food, determine a food processing score based on the vector of probabilities, and output for display the determined food processing score.

A computer-implemented method of identifying a degree of food processing based on food nutrient content includes generating a vector of probabilities based on a nutrient profile for a food. The nutrient profile includes nutrient content data for the food, and each probability of the vector represents a probability associated with a processing category for the food. The method further includes determining a food processing score based on the vector of probabilities and displaying the determined food processing score.

A computer-implemented method of providing a precision nutrition prescription for an individual includes receiving an input comprising an identification of a food for consumption by an individual. The method further includes generating a vector of probabilities based on a nutrient profile for the food and determining a food processing score based on the vector of probabilities. The nutrient profile includes nutrient content data for the food, and each probability of the vector represents a probability associated with a processing category for the food. The method further includes generating a prescription for the individual based on the determined food processing score, the prescription including a recommendation for consumption of the food by the individual, a recommendation for consumption of an alternative food by the individual, or a combination thereof. Optionally, the method can further include receiving an input of biological data relating to the individual, and generating the prescription for the individual can be further based on the received biological data. The method further includes outputting for display the determined prescription,

A precision nutrition engine includes a data source comprising a nutrient profile for each of a plurality of foods. The nutrient profile includes nutrient content data for the food. The precision nutrition engine further includes a processor communicatively coupled to the data source and configured to receive an input comprising an identification of a food for consumption by an individual, generate a vector of probabilities based on the nutrient profile for the food, and determine a food processing score based on the vector of probabilities. The processor is further configured to generate a prescription for the individual based on the determined food processing score and output the prescription for display. The prescription includes a recommendation for consumption of the food by the individual, a recommendation for consumption of an alternative food by the individual, or a combination thereof. Optionally, the engine can further include a data source including biological data of the individual, which can be received by the processor, and the processor can be configured to generate the prescription based further on the received biological data.

A computer-implemented method of providing a precision nutrition prescription for an individual includes receiving an input including an identification of one or more foods consumed by an individual and generating a vector of probabilities based on a nutrient profile for each of the one or more foods. The nutrient profile for each of the foods includes nutrient content data for the food. Each probability of the vector represents a probability associated with a processing category for the food. The method further includes determining a food processing score based on the vector of probabilities for each of the one or more foods, determining an individual food processing score based on the determined food processing scores, and generating a prescription for the individual based on the determined individual food processing score. The prescription includes a recommendation of foods for consumption by the individual. The determined prescription is displayed. Optionally, the method can include receiving an input including biological data of the individual, and the generation of a prescription for the individual can be further based on the received biological data.

A precision nutrition engine includes a data source including a nutrient profile for each of a plurality of foods and a processor communicatively coupled to the data source. The nutrient profile for a food includes nutrient content data for the food. The processor is configured to receive an input comprising an identification of one or more foods consumed by an individual, generate a vector of probabilities based on the nutrient profile for each of the one or more foods, determine a food processing score based on the vector of probabilities for each of the one or more foods, and determine an individual food processing score based on the determined food processing scores. The processor is further configured to generate a prescription for the individual based on the determined individual food processing score, the prescription comprising a recommendation of foods for consumption of the food by the individual, and output for display the determined prescription. Optionally, the engine can further include a data source including biological data of the individual, and the generation of a prescription for the individual can be further based on the received biological data.

The food processing score can be a value representing an orthogonal projection over a line defined by at least two probabilities of the vector. For example, the at least two probabilities of the vector include a probability associated with a processing category representing minimally processed food and a probability associated with a processing category representing maximally processed food. The food processing score (FPS) for a food k can be determined according to:

$${FPS}_{k} = \frac{1 - {p}_{1}^{k} + {p}_{4}^{k}}{2}$$

where p₁^{k} is the probability associated with the processing category representing minimally processed food and p₄^{k} is the probability associated with the processing category representing maximally processed food.

Generating the vector of probabilities can include performing a multi-class random forest classification. The generated vector can include probabilities associated with processing categories corresponding to unprocessed food, culinary ingredient food, processed food, and ultra-processed food, as defined by the NOVA system, or other number or type of processing categories, as per other classification system.

The systems and methods described can further provide for determination of an individual food processing score based on a plurality of determined food processing scores. The individual food processing score can be a weight-based score or a calorie-based score.

For example, an individual food processing score iFPS_{WF}^{j} for an individual j can be determined according to:

$${iFPS}_{WF}^{j} = {\sum }_{k}^{{D}_{j}} \frac{{w}_{k}^{j}}{{W}^{j}} {FPS}_{k}$$

where Dⱼ is a number of dishes consumed by the individual, W^{j} is a daily total amount of consumed food by the individual by weight, and wₖ^{j} is an amount consumed for each food item by weight.

In another example, an individual food processing score iFPS_{WC} for an Individual j can be determined according to:

$${iFPS}_{W C}^{j} = {\sum }_{k}^{{D}_{j}} \frac{{c}_{k}^{j}}{{C}^{j}} {FPS}_{k}$$

where Dⱼ is a number of dishes consumed by the individual, C^{j} is a daily total amount of consumed food by the individual by calories, and cₖ^{j} is an amount consumed for each food item by calories.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a graph illustrating nutrient resolution provided by four food composition databases.

FIG. 2 is a plot of the relative content of sixty nutrients for an example food (onions), with the left side representing onions that are cooked or sautéed from fresh with fat added in cooking and the right side representing onion rings that are prepared from frozen, batter-dipped, and baked or fried.

FIG. 3 is a schematic illustrating an output of probabilities for each of four food processing categories for raw onions (top) and onion rings (bottom) based on nutritional profile data. Each food is represented by a vector of probabilities {p₁}, indicating the likelihood of being classified as unprocessed (NOVA 1), culinary ingredient (NOVA 2), processed (NOVA 3), and ultra-processed (NOVA 4). The dominant probability determines the final classification label (in red).

FIG. 4 is a plot illustrating the classifier results of the manually-classified foods according to the 4-level NOVA classification. Of the foods listed in the USDA Food and Nutrient Database for Dietary Studies (FNDDS), only 34.25% have been manually classified, the results for which are shown in FIG. 4. The classifier comprises a four-dimensional space. A principal component analysis (PCA) was performed, i.e., a mathematical transformation of the original probability features that reduces the number of dimensions of the problem from four to two while providing a visual reconstruction of the data.

FIG. 5 is a plot illustrating classifier results obtained with an example system. The system classified all foods listed in FNDDS and determined that 6.85% of the foods listed in the database are of NOVA Class 1, 0.92% are of NOVA Class 2, 22.82% are of NOVA Class 3, and 69.41% are of NOVA Class 4. The many foods at the boundary regions suggests that confidence in the classification for those foods is not high. The large-dash line (red) represents an example of Eq. 4, described below, on which each food can be orthogonally projected to calculate a Food Processing Score (FPS), as graphically illustrated with the small-dash lines (black) for four example food items.

FIG. 6 is a graph illustrating the ranking of all foods in the FNDDS 2009/2010 database by FPS, as determined by Eq. 1, described below, with example food items identified.

FIG. 7 is a graph illustrating the FPS of the manually-classified NOVA foods.

FIG. 8 is a plot illustrating FPS scores for foods in the Food Categories of What We Eat in America (WWEIA).

FIG. 9 is a graph illustrating consumption patterns based on the FPS of foods in the FNDDS 2015-2016 database and dietary intake data provided by the National Health and Nutrition Examination Survey (NHANES) of foods consumed over two days of dietary interviews. The locations of popular foods are illustrated on the graph, including “fast food pizza with pepperoni” (ranked 6) and “bananas” (ranked 7), which contributed similar amounts to overall daily consumed calories in the U.S. (i.e., 23.67 and 22.89 kcal, respectively) and show a significant difference in processing scores (i.e., FPS_{pizza}=0.9994 and FPS_{banana}=0). To generate the processing scores, the 58-nutrient panel of the FNDDS database was leveraged, and the dietary journals of the 6,875 participants of the NHANES study who completed both dietary interviewers were considered. The results are shown as a Probability Density Function (PDF).

FIGS. 10A-10D show example individual food processing scores (iFPS) and associated data for two example individuals (a, b) from a cohort of 41,474 individuals from four cycles of NHANES (1999-2006). The example individuals (a, b) are two men of 47 and 48 years old and who had similar numbers of dishes and caloric intakes while having different consumption patterns. For each individual in the cohort, the average number of dishes reported in the dietary interviews was determined. FIG. 10A is a graph of a number of consumed dishes over the cohort with dashed lines representing the number of dishes consumed by the example individuals. Individuals (a) and (b) reported 17 and 15 dishes, respectively. For each individual in the cohort, the average daily caloric intake was calculated. FIG. 10B is a graph of daily caloric intake over the cohort with dashed lines representing the caloric intake of the example individuals. Individuals (a) and (b) reported 1,894 and 2,016 kcal, respectively. From the dietary interviews, iFPS scores based on weight (iFPSwc) were derived. FIG. 10C is a graph of iFPSwc scores over the cohort with dashed lines representing the iFPSwc scores of the example individuals. The iFPSwc of individual (a), who consumed mainly simple recipes, was 0.40, and the iFPSwc of individual (b), who consumed more ultra-processed food, was 0.97. FIG. 10D is a chart illustrating the foods consumed by the example individuals (a, b) with associated calories, processing scores (PS) per food, and grams consumed.

FIG. 11 is a graph illustrating association between iFPS and Metabolic Syndrome Risk Factors. Each variable reported on the right (e.g., “Trunk Fat (g)”) is a disease phenotype or a risk factor contributing to “Metabolic Syndrome,” a cluster of conditions that increase the risk of heart disease, stroke, and diabetes. The association of Metabolic Syndrome risk factors was measured with respect to iFPSwF without water consumption, by computing logistic regression for binary values, linear regression for continuous variables, and correcting for age, gender, ethnicity, socio-economic status and caloric intake. The standardized (3 coefficient is reported here, quantifying the effect on each exposure when the Box-Cox transformed diet scores increase of one standard deviation over the population. Each variable is color-coded according to (3, with positive associations in red, and negative associations in blue.

FIG. 12 is a diagram of a process for determining a Food Processing Score (FPS).

FIG. 13 is a diagram of a process for determining an individual Food Processing Score (iFPS).

FIG. 14 is a schematic view of a computer network environment in which embodiments of the present invention may be deployed.

FIG. 15 is a block diagram of computer nodes or devices in the computer network of FIG. 14.

FIG. 16 is a diagram of precision nutrition engine.

### DETAILED DESCRIPTION

In recent years the classification of the food supply has become essential to public health dietary guidelines assisting the population in adopting a healthy diet. NOVA, a popular classification focusing on the extent of food processing, has enabled many epidemiological studies investigating the association between ultra-processed food consumption and disease onset, despite the strong dependence on manual assessment.

Classification processes, such as classification with NOVA, remain limited due to laborious expertise based manual evaluation of each food, which limits coverage. For example, NOVA classification is limited to only 34.25% of foods documented in the National Health and Nutrition Examination Survey (NHANES). The classification process is particularly challenged by composite recipes and products, whose class assignment is not straightforward. Furthermore, the four processing categories defined by NOVA lacks room for nuances, with most manual classifiers choosing to classify all foods with at least one ultra-processed ingredient as ultra-processed, independent of the relative proportion of that ingredient. This is a suboptimal solution to the problem of classifying foods.

A description of example embodiments follows.

Systems and methods are provided that include machine learning processes for efficiently predicting food classification, such as NOVA classification, for food databases with varying nutrient resolution. Other examples of food classification include the NutriScore Labeling System (also referred to as 5-Colour Nutrition Label or 5-CNL), and the traffic light rating system. The systems and methods described were used to assess food items and consumption data provided by the National Health and Nutrition Examination Survey (NHANES) from 1999 to 2016. The systems and methods successfully provided for systematic analysis of several food databases and demonstrated how discrete classification systems, such as NOVA, only partially capture the processing heterogeneity of the food supply. The systems and methods can further provide for determination of a Food Processing Score (FPS), which is based on a continuous index for ranking foods from least processed to most processed. The FPS is not only able to rank food products, but can also be extended to measure an overall quality of an individual's diet, which can provide significant value for epidemiological studies.

The systems and methods provided include a machine learning classifier trained to predict a degree of processing of any food. Food processing can systematically and reproducibly alter a nutrient concentration of food. Using nutrient panels of varying resolution as input, the systems and methods provided can offer nearly perfect predictive performance for current NOVA classes and allow for systematic analysis of the processing state of national databases, such as the USDA Food and Nutrient Database for Dietary Studies (FNDDS) and the USDA National Nutrient Database for Standard Reference (SR), and even grocery store data. By leveraging the decision space of the classifier, a Food Processing Score (FPS) can be determined to indicate a degree of processing of any food. The FPS can enable quantification of diet quality of individuals, as a well as of whole populations of individuals, which can unveil statistical correlations between processed foods and specific disease phenotypes.

As used herein, the term “nutrient” means any chemical entity catalogued by a food composition database. The term “nutrient” includes unique chemicals, such as vitamin C, and aggregate measures, such as total fat and total sugar. For example, a system or method may include selection and consideration of all “nutrients” measured in grams (g), milligrams (mg), micrograms (μg), carried by 100 grams of product.

As used herein, the term “nutrient profile” means a collection of data relating to the nutrient content of a food. A nutrient profile can contain information pertaining one or more nutrients present in a food. For example, a nutrient profile can include nutrient information as is present in a nutrition facts label (e.g., fat, saturated fat, trans fat, cholesterol, sodium, total carbohydrate, dietary fiber, total sugars, added sugars, protein, vitamin C, vitamin D, calcium, iron, potassium). The data included in a nutrient profile can include an amount of the one or more nutrients by weight (e.g., grams), energy (e.g., calories or kilocalories), percent or recommended daily value, or other metric by which nutrient content may be measured, and any combination thereof.

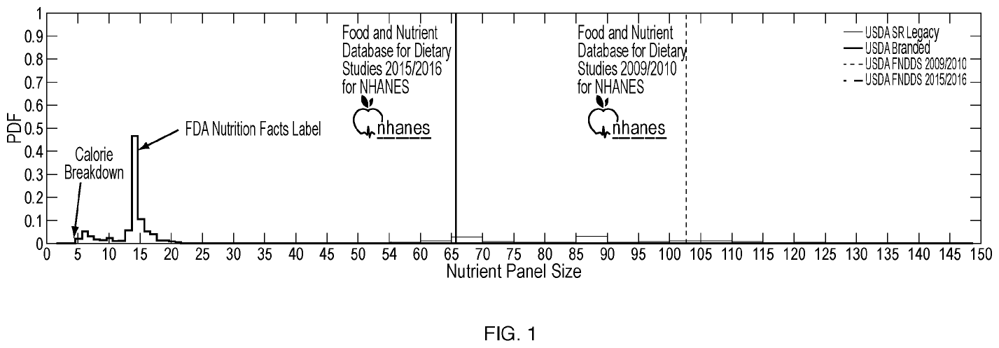

As used herein the term “resolution” with respect to a nutrient profile data means a number of nutrients reported in the profile. For example, USDA SR, an authoritative source of food composition data in the United States, catalogues the nutrient profile of 8,789 foods with resolutions ranging from 8 to 150 nutrients (FIG. 1). In another example, USDA FNDDS, which is designed for epidemiological analysis of dietary intake data collected by NHANES, reports 65 to 102 nutrients for all foods, depending on edition (FIG. 1). Nutrient profile data available to consumers is typically of lower resolution than nutrient profile data available through databases such as SR and FNDDS. For example, the Food and Drug Administration (FDA) mandates the listing of 13 nutrients on a nutrition facts label, which is also an example of a nutrient profile. The 14 nutrients mandated by the FDA for inclusion on a nutrition facts label includes, for example, saturated fat, trans fat, sodium, and vitamin C.

While the FDA mandates the inclusion of 13 nutrients on nutrition facts labelling, branded products are characterized by extreme variability in number of reported nutrients. Approximately 36% of the food supply provides a minimal four nutrient description, including the reporting of calories and a breakdown of the food in total fat, carbohydrates, protein, and alcohol (FIG. 1).

As used herein, the term “food” means any substance consumable by a human or animal that can provide nutrition for maintaining life and growth.

As used herein the term “processing” with respect to a food means alteration of a food from its natural state due to, for example, cooking, packaging, and addition of additives.

As used herein, the term “processing category” with respect to a food or with respect to aggregate foods (e.g., recipes, diets) means a category belonging to an index for categorizing food by extent of processing. For example, NOVA groups foods into four processing categories, including: unprocessed or minimally processed foods (NOVA 1), culinary ingredients (NOVA 2), processed foods (NOVA 3), and ultra-processed products (NOVA 4).

Examples of foods and food types belonging to each of the NOVA processing categories follows. Foods across these categories from the FNDDS and SR databases comprised training data for an example classifier of the provided systems and methods.

Group 1 “unprocessed or minimally processed foods”: fresh, dry or frozen fruits or vegetables, grains, legumes, meat, fish and milk.

Group 2 “processed culinary ingredients”: table sugars, oils, fats, salt, and other substances extracted from foods or from nature, and used in kitchens to make culinary preparations.

Group 3 “processed foods”: foods manufactured with the addition of salt or sugar or other substances of culinary use to unprocessed or minimally processed foods, such as canned food and simple breads and cheese.

Group 4 “ultra-processed foods”: formulations of several ingredients which, besides salt, sugar, oils and fats, include food substances not used in culinary preparations, in particular, flavors, colors, sweeteners, emulsifiers and other additives used to imitate sensorial qualities of unprocessed or minimally processed foods and their culinary preparations or to disguise undesirable qualities of the final product.

NOVA relies upon manual classification, engaging experts to interpret a label for each individual food, which is a time-consuming procedure that has limited its coverage to 2,484 foods in the FNDDS 2009-2010 database, representing only 34.25% of an initial batch of 7,253 items in the database. The remaining 4,769 foods within FNDDS database are either not classified or need further decomposition into ingredients, and hence lack a unique classification and are listed as “Not Classified” or “Composite Recipe” in the database.

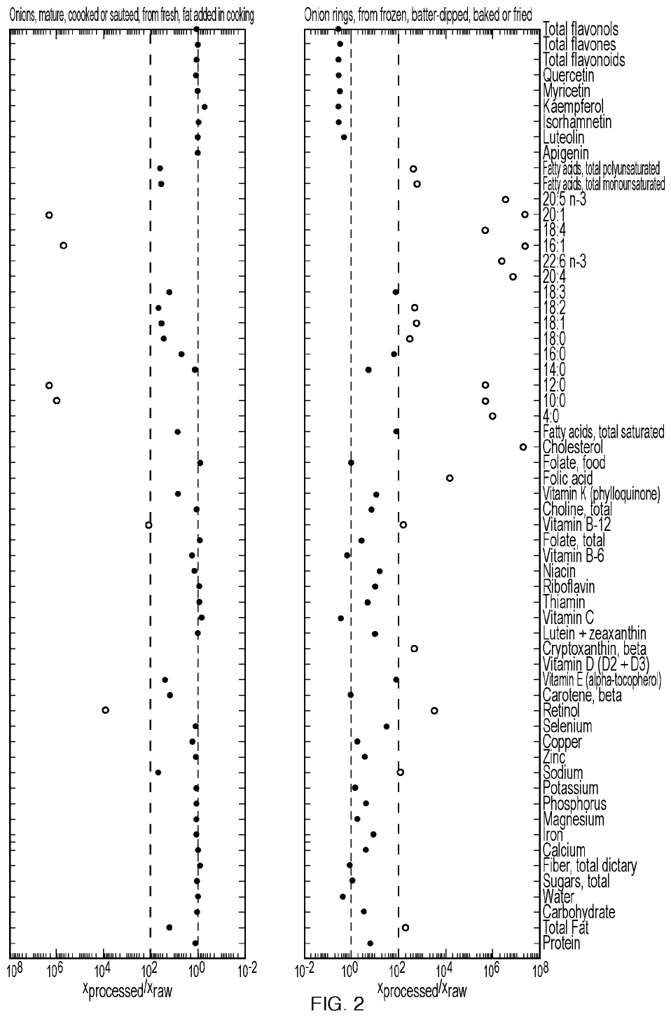

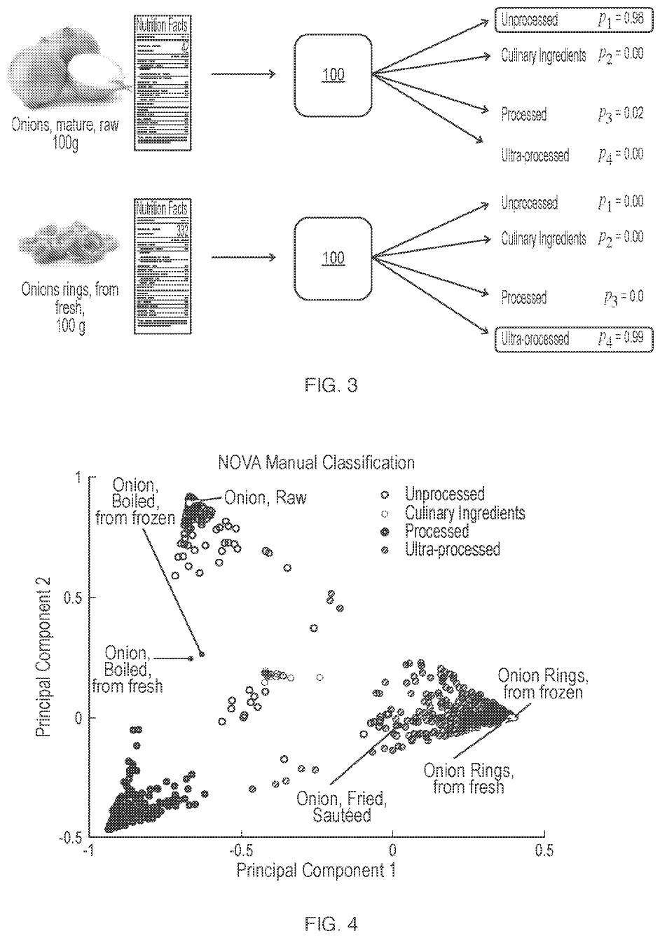

The nutrient composition of food can reflect a physical, biological, and/or chemical process involved in its preparation and conservation. A nutrient profile can provide for unveiling of a degree of processing that a food has undergone during its preparation. For example, changes in the nutrient profile of a raw onion induced by frying and battering are illustrated in FIGS. 2 and 3. It was found that 58.59% of the 99 nutrients recorded in raw onion undergo a change in concentration of more than 10%, and, for 32.32% of the nutrients, like fatty acids 16:1, 20:1, and the flavone apigenin, the change exceeds an order of magnitude. FIG. 2 further illustrates that a single “biomarker” (e.g., a nutrient where concentration alone would indicate a degree of processing) is lacking. Indeed, changes are observed in multiple concentrations whose combinations correlate with processing. This complexity of nutrient variations induced by processing can provide for difficulty in assessing foods to determine a level of processing. Machine learning techniques can efficiently capture a combinatorial explosion of nutrient alterations. Furthermore, while details about food preparation and conservation are rarely available, nutrient composition is relatively easy to access given the multiple food composition databases (e.g., the databases profiled in FIG. 1).

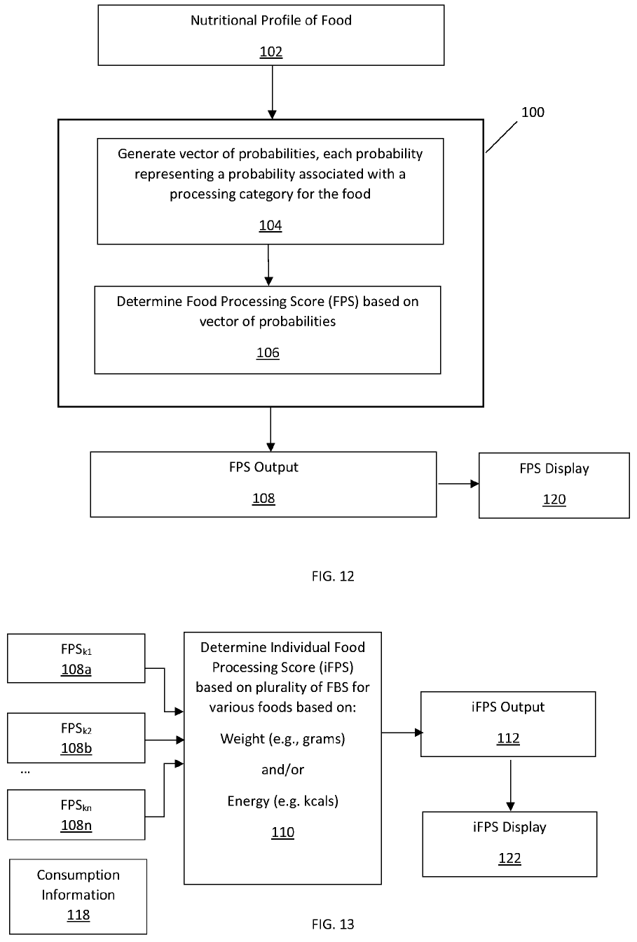

A method 100 of identifying a degree of food processing based on food nutrient content is illustrated in FIG. 12. As illustrated, an input 102 comprising a nutritional profile of a food is provided, from which a vector of probabilities 104 is generated. Each probability of the vector represents a probability associated with a processing category for the food. The method 100 further includes determining a food processing score (FPS) 106 based on the vector of probabilities. An output 108 representing the determined FPS can be provided. For example, the FPS output 108 can be provided for further processing, such as for determination of an iFPS (FIG. 13), or can be provided as a display 120.

The generation of a vector of probabilities {pᵢ} can include classification with a machine learning technique, such as a random forest classifier, gradient boosting framework (e.g., XGBoost), Naïve Bayes classifier, support vector machine, and artificial neural network. For example, a system executing the method 100 can include a multi-class random forest classifier configured to predict a processing level of a food from a nutrient profile of the food (FIG. 3). As illustrated in the example shown in FIG. 3, the vector of probabilities {pᵢ} includes probabilities representing the likelihood that the food is classified as unprocessed (p₁, NOVA 1), culinary ingredient (p₂, NOVA 2), processed (p₃, NOVA 3), and ultra-processed (p₄, NOVA 4). The highest of the four probabilities determines a final classification label for the food item.

The classifier probability space is a 4D probability simplex that collects all vectors satisfying:

{\vec{p}}∈ [symbol] ⁴,p₁+p₂+p₃+p₄=1,pᵢ≥0∀ᵢ (1)

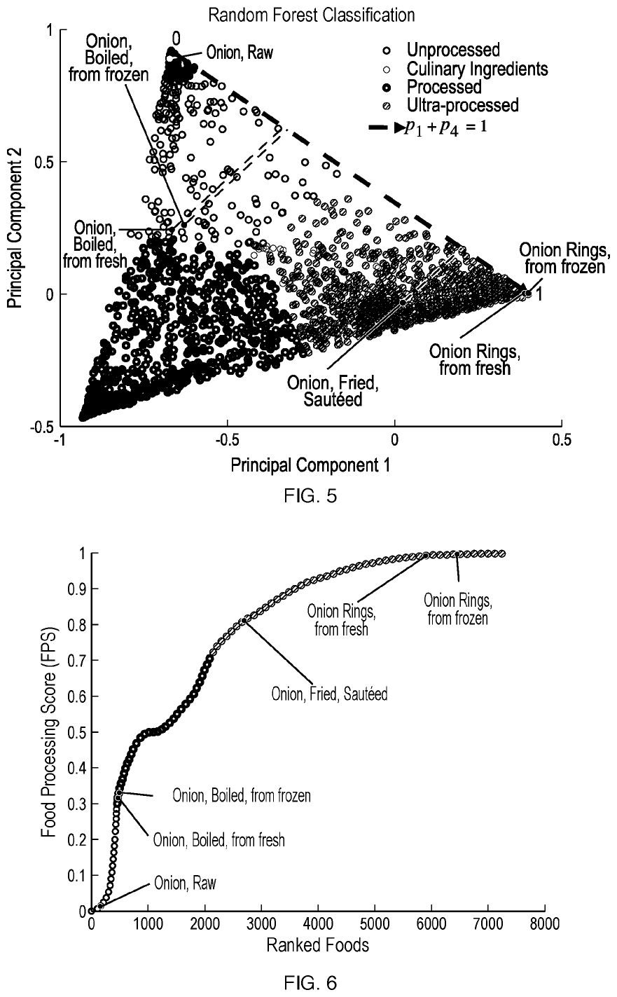

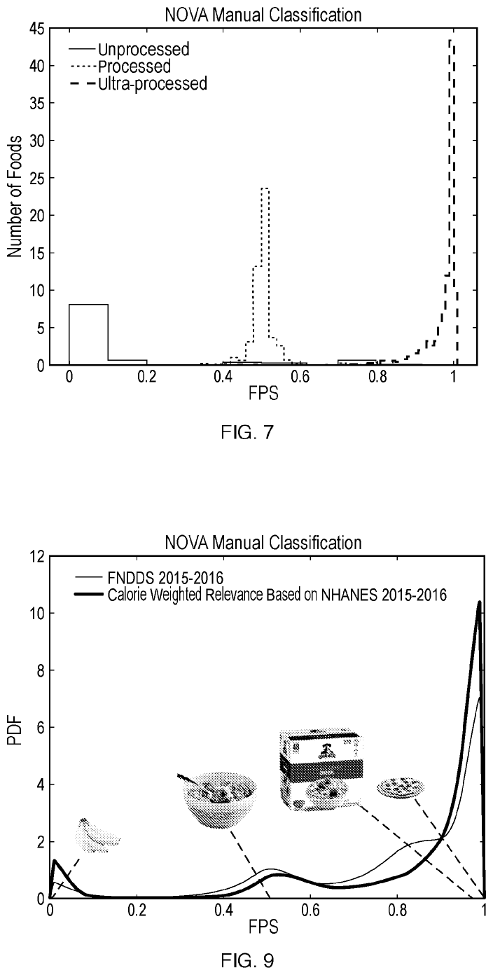

As described further in Example 1 below and shown in FIGS. 4 and 5, the discrete classes cause ambiguities in food classification. A FPS can address this issue by providing a continuous variable, whose value is zero for raw ingredients (FPS=0) and which converges to FPS=1 for ultra-processed foods. The gradual scale overcomes ambiguities observed at the boundaries of the four NOVA classes, where the classifier is forced to choose between classes with largely indistinguishable nutrient profile and probabilities (FIG. 7).

The FPS for a food k is defined as the orthogonal projection:

\vec{pₖ}=(p₁^{k},p₂^{k},p₃^{k},p₄^{k}) (2)

over the line p₁+p₄=1, or as:

$${FPS}_{k} = \frac{1 - {p}_{1}^{k} + {p}_{4}^{k}}{2} . \qquad ( 3 )$$

The projection of any food \vec{pₖ} over the line going from the pure minimally-processed state \vec{p}_{MP}=(1, 0, 0, 0) to the pure ultra-processed state \vec{p}_{UP}=(0, 0, 0, 1), represented by the parametric equation:

$$\vec{l ( t )} = [ \begin{matrix}1 \\ 0 \\ 0 \\ 0\end{matrix} ] + t [ \begin{matrix}- 1 \\ 0 \\ 0 \\ 1\end{matrix} ] , \qquad ( 4 )$$

equivalent to the explicit equation p₁=1−p₄ The projection of food \vec{pₖ} follows as the intersection between Eq. 4 and the plane passing through \vec{pₖ} and orthogonal to \vec{l(t)}, i.e.

−p₁+p₄+p₁^{k}−p₄^{k}=0. (5)

The parameter t* satisfying Eqs. 4 and 5 determines the processing score FPSₖ in Eq. 3.

Eq. 3 can correctly capture the progressive alteration of nutrient content determined by processing, as illustrated by the increasing FPS for onion products shown in FIG. 6, from raw onion (FPS=0.0125) to boiled (FPS=0.3150), fried onion (FPS=0.8121). and onion rings from frozen ingredients (FPS=0.9978). The functional dependence of Eq. 3 on p₁ and p₄ is optimized to distinguish unprocessed from ultra-processed food and assigns all foods with p₂ or p₃≈1 a processing score close to 0.5, i.e., an intermediate level of processing equidistant from pure unprocessed and ultra-processed foods, as Eq. 3 is optimized to distinguish unprocessed from ultra-processed food.

While the classifier and FPS equations are described above with respect to the NOVA system and its four-class categorization, it should be understood that similar classifier spaces and food processing scores can be provided for other classification systems. For example, an established food processing classification system may provide for more or fewer classes (e.g., 3, 5, 6 or 10) as opposed to the NOVA four class system. The methods and systems described can be adapted to accommodate fewer or more class categorizations.

As noted in Example 1 below, it has been found that 69% of the food supply consists of ultra-processed food (NOVA 4). To provide for an understanding of the degree at which ultra-processed foods are present in one's diet, a determination of an individual Food Processing Score (iFPS) can be provided.

A method 110 of determining an iFPS is shown in FIG. 13. A plurality of FPS outputs (108a, 108b . . . 108n), as from method 100, can be provided together with consumption data 118 pertaining to calories or weight consumed of each food by an individual for determination of an iFPS. For example, the iFPS can be a weight-based score or an energy-based score. An output 112 representing the determined iFPS can be provided. The iFPS output 112 can be provided for further processing, such as for further determination of average iFPS scores across a population, or can be provided as a display 122.

A weight-based iFPS_{WF}^{j} for an individual j can be determined according to:

$${iFPS}_{WF}^{j} = {\sum }_{k}^{{D}_{j}} \frac{{w}_{k}^{j}}{{W}^{j}} {FPS}_{k} \qquad ( 6 )$$

where Dⱼ is a number of dishes consumed by the individual, W^{j} is a daily total amount of consumed food by the individual by weight, and cₖ^{j} is an amount consumed for each food item by weight.

An energy-based iFPS_{WC}^{j} for an individual j can be determined according to:

$${iFPS}_{W C}^{j} = {\sum }_{k}^{{D}_{j}} \frac{{c}_{k}^{j}}{{c}^{j}} {FPS}_{k} \qquad ( 7 )$$

where Dⱼ is a number of dishes consumed by the individual, C^{j} is a daily total amount of consumed food by the individual by calories, and cₖ^{j} is an amount consumed for each food item by calories.

While iFPS has been described with respect to a diet of an individual, an iFPS score can be applied for other aggregate measurements, such as for a recipe comprising a plurality of ingredients, for a meal comprising a plurality of dishes, and for foods consumed by a plurality of individuals or by a population of people.

Test systems and methods for determining a degree of food processing were evaluated, the results for which are described in Examples 1-4 herein.

The test systems and methods included a random forest classifier that predicts the processing class of any food, using a reported nutrient panel for the food as input. The excellent agreement between the predictions and the existing manual classification suggests that each of the NOVA classes correspond to clear patterns of nutrient alterations, that are not captured by a single biomarker, but represent combinatorial patterns accurately captured by machine learning. The machine learning approach also inspired a continuous Food Processing Score (FPS), that helps an investigation of how processing modulates the nutrient content of our food. Its extension to measure of the overall quality of an individual's diet showed predictive power over several health phenotypes, confirming and expanding the outcomes of previous studies that successfully linked the consumption of ultra-processed food to disease onset. Additionally, the computation of FPS can easily adapt to different sets of nutrients, allowing for the accurately classification of food even from limited nutrient information. With nutrition facts becoming easily accessible to consumers via smartphone apps, web portals, and grocery store websites, the food processing score FPS can help guide making individual choices, and to monitor the reliance of an individual's eating pattern on processed and ultra-processed food.

The resolution of existing food databases can be limited. Indeed, many chemicals like acrylamide, ammonium sulfate, azodicarbonamide, butylated hydroxyanisole, and furans, associated with different steps of preparation and preservation of food, are currently not tracked by national agencies. The lack of quantification of these chemicals becomes even more striking once the body of scientific literature devoted to impact on human health is acknowledged. Our analysis shows that an unsupervised hierarchical clustering of foods, leveraging the current nutrient panels, is not able to independently reproduce the four NOVA classes. It is possible, however, that the addition of chemical measurements that pertain to processing signatures can further improve the current result, leading to improved chemically-driven classification of food processing.

The test system and methods providing for the food processing score is inclusive of an entire documented food supply, discriminating between unprocessed and ultra-processed food. The systems and methods provided can also be applied to discriminate among foods within specific classes of interest. For instance, an FPS can be optimized for products collectively classified as ultra-processed (NOVA 4), which can enable researchers and health professionals to create healthier alternatives to the most highly-consumed ultra-processed foods, with more balanced chemical composition.

Beyond the analysis of single food items, the introduction if the iFPS, a processing score characterizing the diet of each individual was also provided and evaluated. Different from other dietary indexes, such as REI-15, designed to measure alignment of individuals' diets with the 2015-2020 Dietary Guidelines for Americans, the interplay between the iFPS and FPS advantageously provides for identification of those foods to target to shift individual consumption towards a less processed diet, offering an informed choice over products belonging to the same food category.

Systems and methods described herein can provide for automatic assessment of the processing level of any food, with information conveyed to a user through display a FPS and/or iFPS. The systems and methods described can be applied to analysis of entire food supplies and monitor changes in food supply over time, which can be advantageous for public health assessment and monitoring. Furthermore, iFPS can provide for evaluation of dietary intake of processed foods for individuals, which can be paired with other health data as described above to monitor health. The display of FPS and iFPS outputs can be useful directly for users, but can also be displayed in combination for multiple food items as a recommendation tool for a user. For example, a plurality of FPS scores can be displayed to provide a comparison of the processing level of multiple foods. In a further example, a user may obtain the FPS score for a given food (e.g., ketchup) and the display may provide information for the selected brand and item with FPS scores of similar food items from the same or different brans such that the user can make an informed choice as to a less-processed product.

In a further example, the FPS ca be provided a recommendation tool for suggesting cooking and/or preserving methodologies that minimally alter raw ingredients. Recipes can also be provided with FPS output, and recipes can be tested to determine which recipe variations permit for the production of a least or lesser-processed food product.

The systems and methods described can provide for precision nutrition recommendations on an individual basis. For example, the systems and methods can be used to prescribe one or more foods to an individual.

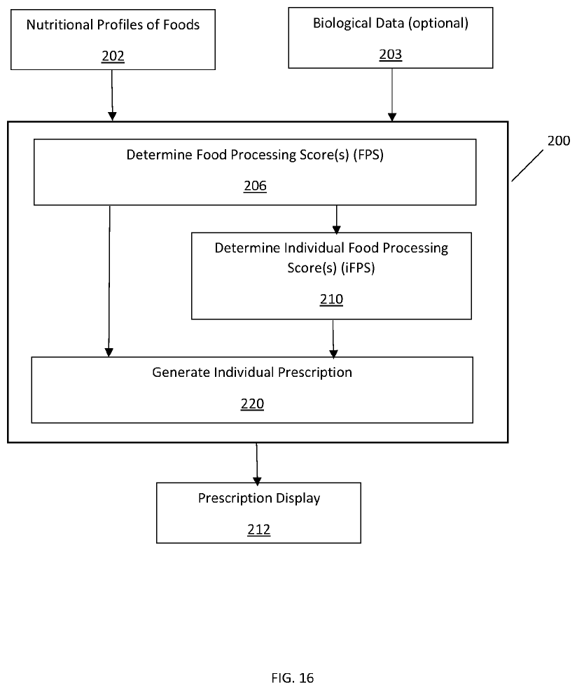

An example of a precision nutrition engine 200 is shown in FIG. 16. Nutritional profiles of one or more foods 202 are provided, optionally along with biological data 203 for an individual. The engine determines one or more food processing scores (FPSs) 206 and generates an individual prescription 220. The prescription 220 can be provided on an individual food basis or on a diet basis. For example, the nutritional profile provided and the determined food processing score can be for a food for consumption by an individual. The prescription can include a recommendation for consumption of the food by the individual, a recommendation for consumption of an alternative food by the individual, or a combination thereof. Alternatively, or in addition, the nutritional profiles provided can be provided for a plurality of foods that were consumed by the individual for the purpose of monitoring and tailoring a diet for the individual. The engine can then determine an individual food processing score, and the prescription can include a recommendation of foods for consumption by the individual so as to maintain or modify the individual's diet.

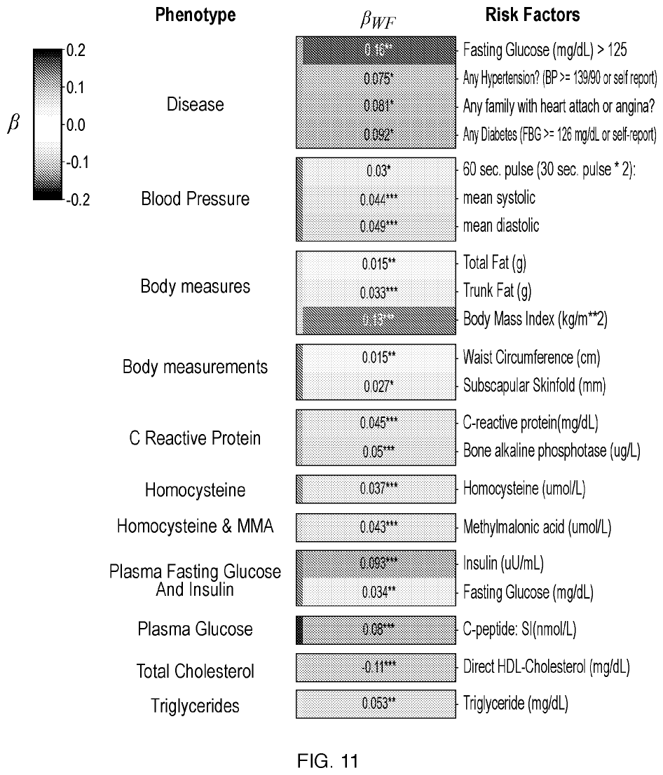

Optionally, biological data can be included. For example, biological data relating to risk factors for cardiovascular diseases, hypertension, and diabetes can be provided to the engine and used in conjunction with the determined FPS and/or iFPS for generating a prescription for the individual. Examples of biological data include blood pressure, body measurements, and blood analysis, as shown in FIG. 11. In an example prescription, an individual with biological data indicating an increased risk of disease may receive a more conservative prescription of foods with lower FPS than an individual without risk factors.

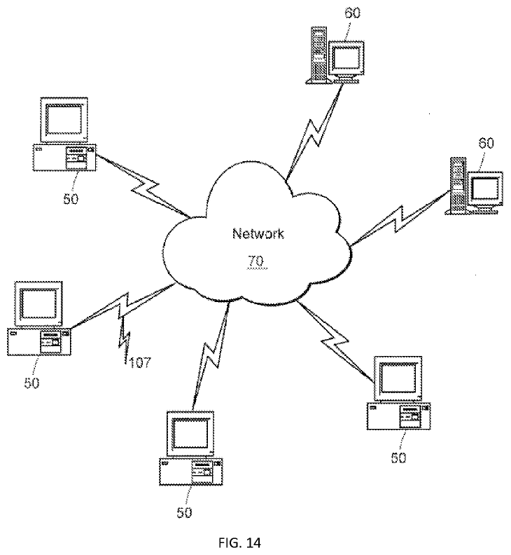

FIG. 14 illustrates a computer network or similar digital processing environment in which the systems and methods described may be implemented. Client computer(s)/devices/exercise apparatuses 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), a worldwide collection of computers, cloud computing servers or service, Local area or Wide area networks, and gateways that currently use respective protocols (TCP/IP, Bluetooth, etc.) to communicate with one another. Other electronic device/computer network architectures are suitable.

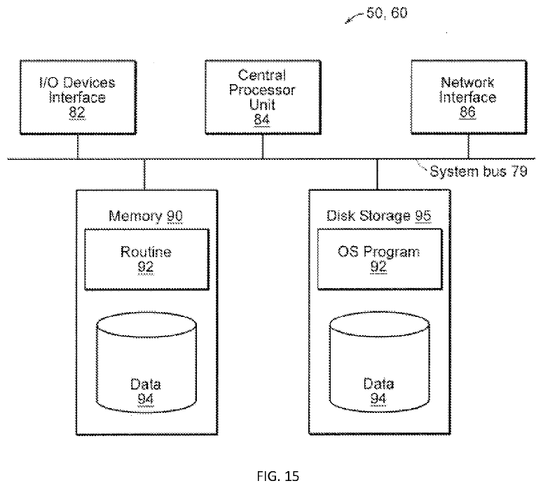

FIG. 15 is a diagram of the internal structure of a computer (e.g., client processor/device 50 or server computers 60) in the computer network of FIG. 14. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 3). Memory 90 provides volatile storage for computer software instructions 92 and data 94 used to implement embodiments of the present invention (e.g., processor routines and code for creating a directed acyclic graph (DAG) as a function of computed alignment indices and aligning sequence reads against the DAG being developed, as described herein). Disk storage 95 provides nonvolatile storage for computer software instructions 92 and data 94 used to implement an embodiment of the present invention. Central processor unit 84 is also attached to system bus 79 and provides for the execution of computer instructions.

In particular, embodiments of the present invention execute processor routines for the methods 100, 110 of FIGS. 12 and 13, respectively. In one embodiment, the processor routines 92 and data 94 are a computer program product (generally referenced 92), including a non-transitory computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, other mediums and the like.

In other embodiments, the computer program product 92 provides Software as a Service (SaaS) or similar operating platform.

Alternative embodiments can include or employ clusters of computers, parallel processors, or other forms of parallel processing, effectively leading to improved performance, for example, of generating a computational model. Given the foregoing description, one of ordinary skill in the art understands that different portions of processor routine 100, 110 and different iterations operating on respective sequence reads may be executed in parallel on such computer clusters or parallel processors.

### EXEMPLIFICATION

### Example 1: Classifier Training and Performance

A classifier, alternatively referred to as FoodProX, is a multi-class random forest classifier that accepts as input a reported nutrient panel of a food and predicts a processing-class of the food.

A multi-class random forest classifier was trained to automatically predict the processing level of any food, given its logarithmic nutrient profile, with the goal to classify all those foods in FNDDS not included in the 4-level classification described herein. The majority of the unclassified foods (4,039) were treated as composite dishes, i.e. food items that remained to be decomposed into ingredients to classify separately. The remaining part of the database is composed by 730 food items, present in FNDDS but never taken into account by the analysis. The logarithmic value corresponding to zero, i.e. “absence of a nutrient”, was set to −20, by observing the distribution of non-zero values of the entire database.

The classification problem is strongly unbalanced, given the high number of items in classes 3 and 4, compared to class 1 and 2. To address this issue, the final version of the classifier was trained with SMOTE (Chawla N V., Bowyer K W, Hall L O, Kegelmeyer W P. SMOTE: Synthetic minority over-sampling technique. J Artif Intell Res. 2002; 16(1):321-357), a resampling technique that increases the sensitivity of a classifier to the minority classes.

A 5-fold cross validation (without SMOTE) over the labeled database was performed, obtaining excellent performance: AUC over the four classes (0.9806±0.0028 for NOVA1, 0.9880±0.0104 for NOVA2, 0.9649±0.0082 for NOVA3, 0.9768±0.0048 for NOVA4); and AUP over the four classes (0.8885±0.0138, 0.7962±0.0670, 0.8821±0.0367, 0.9924±0.0027).

Additionally, for all the foods the random forest computer-implemented method returns the likelihood to belong to each one of the four classes {pᵢ}, encoding the fraction of trees in the ensemble voting for a given class. When p₁, the likelihood of being unprocessed, is dominant over p₂, P₃ and p₄ the food is classified as unprocessed. However, by inspecting the continuous distribution of p₁ it can quantify how different types of processing alter the initial raw ingredients and progressively decrease the likelihood of the food to be unprocessed.

In a second implementation to train the classifier, 2,484 foods classified by NOVA were provided as input and used to learn nutrient patterns associated with food processing, enabling the classifier to automatically classify any food into the NOVA processing categories. The results obtained from the classifier for the manually-classified NOVA foods is shown in FIG. 4. The classifier was able to identify mistakes in the manual-classifications.

The classifier was applied to 7,253 foods listed in the FNDDS database for which an extended nutritional panel quantifying the presence of 99 nutrients expressed in grams (g), milligrams (mg), and micrograms (m) carried by 100 grams of food product was available.

It was found that, by relying on the reported nutrients, the model ranks 98% of the time a true unprocessed food (NOVA 1) higher than a randomly selected processed food (NOVA 3,4), easily separating the unprocessed food from other categories (AUC=0.98). Little difference in the performance of the classifier was found; the AUC values were consistently high for each of the four NOVA classes (0.9806±0.0028 for NOVA1, 0.9880±0.0104 for NOVA2, 0.9649±0.008 2 for NOVA3, 0.9768±0.0048 for NOVA4), and far from a random performance with AUC=0.5, describing a model with no discriminative power. The stable performance of the classifier demonstrated that changes in the nutrient content of food has significant predictive power when it comes to ascertain the extent of food processing, confirming the existence of a strong association between processing and the nutrient profile.

FoodProX was used to classify all foods, whether or not the foods had (34.25%) or lacked (65.75%) NOVA classification, finding that 6.85% of the full FNDDS database consists of NOVA 1, 0.92% of NOVA 2, 22.82% of NOVA 3, and 69.41% of NOVA 4 foods (FIG. 5). For each food in FNDDS, the likelihood of belonging to each of the four classes {p₁} was analyzed, summarizing the confidence of the model in taking the respective decision (FIG. 3). The analysis of these continuous probabilities indicates that 83.19% of the manual labels correspond to foods with a single dominant probability (p₁ >0:90), i.e., can be confidently assigned to one of the four NOVA classes. Yet, 16.81% of foods lack a dominant probability, mainly because they correspond to composite foods and recipes (FIG. 4). If the decision space of the classifier is visualized by performing a principal component analysis over the probabilities {p₁}, it is observed that the manual classification offered by NOVA is largely limited to the three corners of the phase space, to which the classifier assigns dominating probabilities (FIG. 4). Yes, as FIG. 5 shows, many of the previously not classified foods lack a dominating probability, being scattered inside the phase space. This representation allowed for direct observation of an extended boundary region, populated by foods whose assignment in one of the NOVA classes is somewhat arbitrary.

FoodProX automatically detects these boundaries as low confidence in the classification (FIG. 5). The existence of these boundaries is not an inherent limitation of FoodProX, but reflects the fact that a four-class classification defined by NOVA does not accurately capture the nutrient variability characterizing some cooking and processing methods. For example, the classifier assigns “Raw Onion” to NOVA 1 with a dominant p₁=0.977, and with a similar confidence, it assigns “Onion rings prepared from frozen” (p_{4=0.997}) and “Onion rings prepared from fresh” (p₄=0.989) to NOVA 4. In contrast, the classifier offers a lower confidence in classifying “Onion, Sautéed” as NOVA 4, placing it with probability p₄=0.701 in this class and with probability p₃=0.221 in NOVA 3.

### Example 2. Evaluation of Food Processing Score (FPS)

To test the discriminatory power of FPS, the FPS for all foods manually classified by NOVA was measured. As indicated in FIG. 6, all manually labeled unprocessed foods (NOVA 1) have a narrow FPS in the vicinity of 0.1, and all ultra-processed foods (NOVA 4) have an FPS between 0.9 and 1, indicating the ability of the FPS to easily distinguish these two classes, together representing 80% of the food supply. The remaining items, where FPS fluctuates around 0.5 are NOVA 3 items, made by adding sugar, oil, salt, or other culinary ingredients to NOVA 1 products, as well as preserved products or the outcome of non-alcoholic fermentation, and are clearly separated from NOVA 1 and NOVA 4. The FPS allowed for unveiling a degree of food processing characterizing different food preparation techniques, providing lower scores to foods made from fresh ingredients than those made from frozen ingredients (FIG. 6).

It was found that 73% of the foods are ultra-processed. Yet, foods in this category do show different degrees of food processing, some representing composite recipes that contain a minimal amount of ultra-processed ingredients, while others being a result of massive ultra-processing, like chocolate-coated fudge and barbecue sauce. As over 60% of the calorie intake in the U.S. population relies on ultra-processed foods, the distinction can be important. The FPS enables for the distinction of such foods in this category.

### Example 3. Assessment of Processed Food in the American Diet

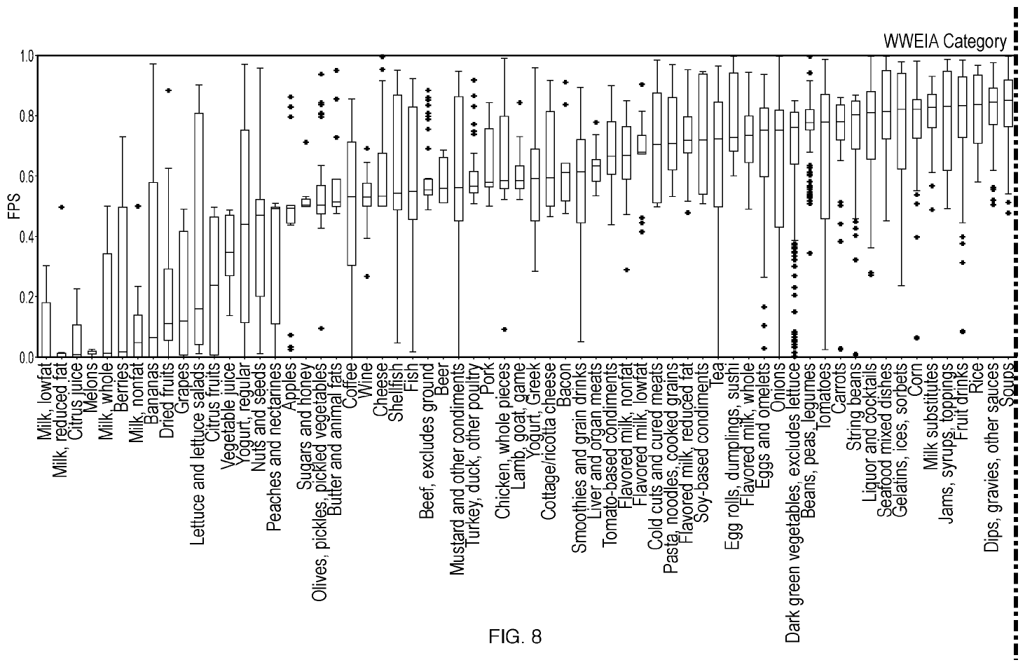

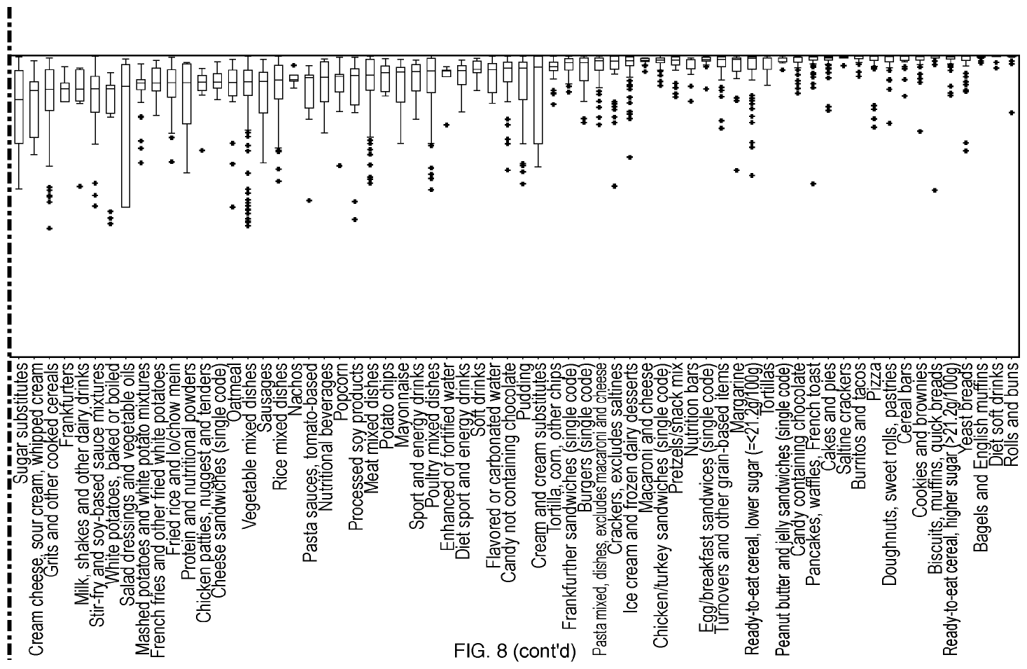

To assess to what degree ultra-processed foods are present in the American diet, data available through NHANES 2015-2016, which includes data from 2-day dietary interviews capturing the dietary choices of 5,266 individuals chosen to be representative of the US population, was analyzed. As indicated the blue curve of FIG. 9, US food consumption is dominated by ultra-processed food, appearing as a major peak near FPS 1. When each item is weighed according to its contribution to the caloric intake, an even higher peak at FPS 1 is observed (red line), indicating that when an amount consumed is factored in, the caloric contribution of the ultra-processed food is even higher. Two smaller peaks at FPS 0.5 and 0.8 are also observed, discriminating between fried food (FPS 0.8), or foods cooked in significant amounts of plant and animal fats, and simpler recipes (FPS 0.5). These peaks are reduced once distribution is normalized by caloric intake. Overall, it was found that the average caloric intake of Americans is dominated by ultra-processed foods combined with a few fruits. For instance, if foods are sorted according to their average caloric contribution to the American diet, it is found that “bananas” rank 7^{th} and provide 22.89 kcal, which is close to “fast-food pizza with pepperoni,” which is ranked 6^{th} and provides 23.67 kcal, yet a significant difference in processing score exists between the two foods (FPS_{banana}=0, FPS_{pizza}=0.9994). Moreover, among products belonging to the same food category a classified by “What We Eat in America” (WWEIA), significant variability in FPS is observed. For example, a breakfast stable food as “oatmeal” ranges from FPS=0.5010 for plain multigrain oatmeal to FPS=0.9881 for an instant, fruit-flavored version of oatmeal cooked with fats (FIGS. 8 and 9).

### Example 4. Individual Food Processing Scores

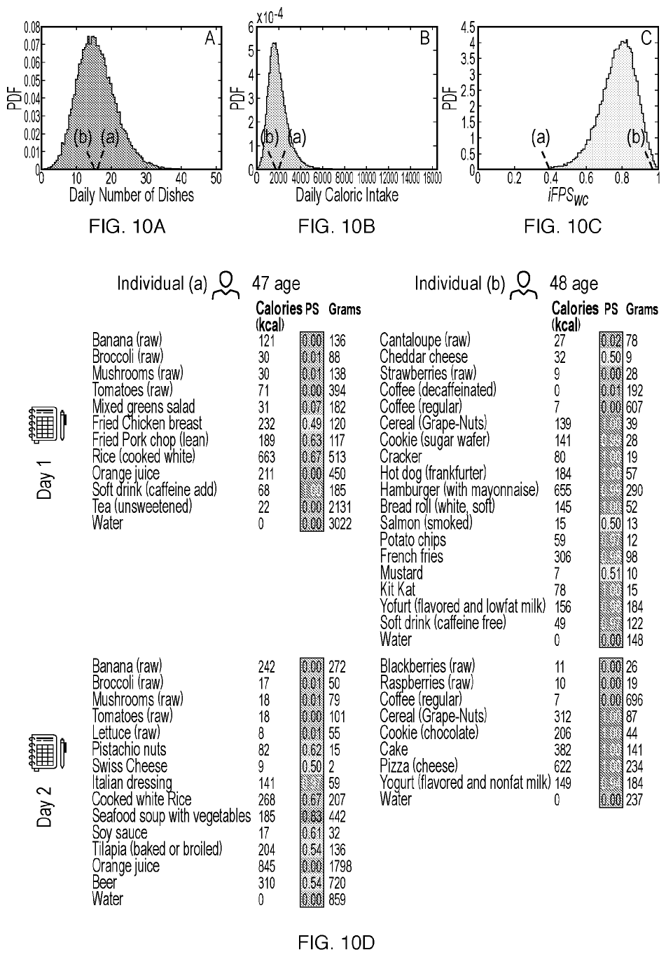

For each individual with dietary records, the diet processing scores iFPS_{WF}^{j}, iFPS_{WC} were calculated for the pooled cohort of 20,046 individuals in NHANES 1999-2006. As FIG. 10C shows, the iFPS of the American population ranges between 0.10, corresponding to diets heavy on raw and home cooked ingredients, to 0.99, capturing diets dominated by ultra-processed food. The distribution is peaked at iFPS 0.78, indicating a high reliance of the American caloric intake on ultra-processed food. We find that iFPS successfully distinguishes between eating patterns of different reliance on processed food. Consider for instance individual (A) and (B) whose two-day diet is shown in FIG. 10D, both being men of similar age (47 vs. 48 years old), with similar number of reported dishes (17 vs. 15 dishes) and comparable caloric intake (2,016 vs. 1,894 kcal). Yet, these two individuals have rather different reliance on ultra-processed food: the diet of individual (A) has iFPS≈0.3971, representing a diet relying on unprocessed ingredients and home cooking. Indeed, half of the calories of individual (A) come from orange juice, rice cooked with no fat, and chicken breast fried with no coating. In contrast, for (B) iFPS=0.9677, as he derives 50% of his caloric intake from ultra-processed foods like pizza with cheese topping, hamburger with mayo and catsup, and ice-cream cake. These different consumption patterns places them in the two opposite sides of the population-based iFPS distribution (FIGS. 10A-10C).

The ability to quantify the reliance of each individual's diet on processed food enables an examination of a degree to which the consumption of processed and ultra-processed food correlates with health outcomes. From the over 1,000 exposures and phenotypes provided in NHANES, the following study was limited to those with a clear connection to diet to avoid confounding factors. For each variable, an association with diet processing scores iFPS was measured by computing logistic regression for binary values, and linear regression for continuous variables, and correcting for age, gender, ethnicity, socio-economic status and caloric intake. After False Discovery Rate (FDR) correction for multiple testing, 194 variables survived, allowing for determination of when and how high iFPS values affect health. The results are shown in FIG. 11, which reports the association of iFPS_{WF} with exposures contributing to Metabolic Syndrome, a biochemical phenotype determined by a group of factors that increase the risk for heart disease, diabetes, and stroke. It was found that high levels of iFPS_{WF}^{j} are significantly associated with an increased risk for cardiovascular diseases, hypertension, and diabetes, in line with the findings reported in Nardocci and in De Deus Medonça (Nardocci, M., Polsky, J. Y. & Moubarac, J. C. Consumption of ultra-processed foods is associated with obesity, diabetes and hypertension in Canadian adults. Canadian Journal of Public Health 1-9 (2020); De Deus Medonça et al., Ultra-processed food consumption and the incidence of hypertension in a mediterranean cohort: The seguimiento universidad deunavarra project. American Journal of Hypertension 30, 358-366 (2017)). Individuals with a high iFPS, indicative of a higher consumption of processed food, exhibit higher blood pressure and, overall, higher scores in several indicators, such as Body Mass Index (BMI) (in agreement with Poti, J. M., Braga, B. & Qin, B. Ultra-processed Food Intake and Obesity: What Really Matters for Health-Processing or Nutrient Content? 6, 420-431 (2017)), trunk fat, and subscapular skinfold.

The modules related to blood panel analysis indicate that high values of iFPS correlate with higher values of fasting glucose and insulin in blood serum and plasma, lower “good” cholesterol HDL, and higher level of triglycerides. Further, novel findings among metabolites' alterations are indicative of an increased risk of type 2 diabetes (C-peptide), inflammation (C-Reactive Protein), heart disease, vitamin deficiency (Homocysteine, Methylmalonic acid), and metabolic bone diseases (Bone alkaline Phosphatase). Strikingly, a negative association between iFPS_{WC}^{j} and telomere length, a biomarker for biological age that is known to be affected by diet through inflammation mechanisms and oxidation, was found, suggesting a higher biological age for individuals relying on highly processed diet, confirming the results shown in Alonso-Pedrero, L. et al. (Alonso-Pedrero, L. et al. Ultra-processed food consumption and the risk of short telomeres in an elderly population of the Seguimiento Universidad de Navarra (SUN) Project. The American journal of clinical nutrition 111, 1259-1266 (2020)).

Furthermore, it was found that a diet rich in highly processed food shows association with increased quantities of carcinogenic compounds like benzenes (abundant in soft drinks), furans (common in many canned and jarred foods), polychlorinated biphenyls (linked to processed meat products such as hot dogs), and perfluorooctanoic acids (found in the wrappers of some fast foods, microwavable popcorn, and candy wrappers), all compounds currently not reported in food composition databases, but recovered at the population level in blood and urine panels.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. (canceled)

2. The computer-implemented method of claim 23, wherein the food processing score is a value representing an orthogonal projection over a line defined by at least two probabilities of the vector.

3. The computer-implemented method of claim 2, wherein the at least two probabilities of the vector include a probability associated with a processing category representing minimally processed food and a probability associated with a processing category representing maximally processed food.

4. The computer-implemented method of claim 23, wherein the food processing score FPSₖ for a food k is determined according to:
$${FPS}_{k} = \frac{1 - {p}_{1}^{k} + {p}_{4}^{k}}{2}$$
where p₁^{k} is the probability associated with the processing category representing minimally processed food and p₄^{k} is the probability associated with the processing category representing maximally processed food.

5. The computer-implemented method of claim 23, wherein generating the vector of probabilities includes performing a multi-class random forest classification.

6. The computer-implemented method of claim 23, wherein the vector includes probabilities associated with processing categories corresponding to unprocessed food, culinary ingredient food, processed food, and ultra-processed food.

7. The computer-implemented method of claim 6, wherein the processing categories are as defined by the NOVA food classification system.

8. The computer-implemented method of claim 1, wherein the method further includes determining an individual food processing score based on a plurality of determined food processing scores.

9. The computer-implemented method of claim 8, wherein the individual food processing score is a weight-based score or a calorie-based score.

10. The computer-implemented method of claim 8, wherein the individual food processing score iFPS_{WF}^{j} for an individual j is determined according to:
$${iFPS}_{W F}^{j} = {\sum }_{k}^{{D}_{j}} \frac{{w}_{k}^{j}}{{W}^{j}} {FPS}_{k}$$
where Dⱼ is a number of dishes consumed by the individual, W^{j} is a daily total amount of consumed food by the individual by weight, and wₖⁱ is an amount consumed for each food item by weight.

11. The computer-implemented method of claim 8, wherein the individual food processing score iFPS_{WC}^{j} for an individual j is determined according to:
$${iFPS}_{W C}^{j} = {\sum }_{k}^{{D}_{j}} \frac{{c}_{k}^{j}}{{C}^{j}} {FPS}_{k}$$
where Dⱼ is a number of dishes consumed by the individual, C^{j} is a daily total amount of consumed food by the individual by calories, and cₖ^{j} is an amount consumed for each food item by calories.

12. (canceled)

13. The system of claim 25, wherein the food processing score is a value representing an orthogonal projection over a line defined by at least two probabilities of the vector.

14. The system of claim 13, wherein the at least two probabilities of the vector include a probability associated with a processing category representing minimally processed food and a probability associated with a processing category representing maximally processed food.

15. The system of claim 25, wherein the processor is configured to determine the food processing score FPSₖ for a food k is determined according to:
$${FPS}_{k} = \frac{1 - {p}_{1}^{k} + {p}_{4}^{k}}{2}$$
where p₁^{k} is the probability associated with the processing category representing minimally processed food and p₄^{k} is the probability associated with the processing category representing maximally processed food.

16. The system of claim 25 wherein the processor is configured to perform a multi-class random forest classification to generate the vector of probabilities.

17. The system of claim 25, wherein the vector includes probabilities associated with processing categories corresponding to unprocessed food, culinary ingredient food, processed food, and ultra-processed food.

18. The system of claim 17, wherein the processing categories are as defined by the NOVA food classification system.

19. The system of claim 25, wherein the processor is further configured to determine an individual food processing score based on a plurality of determined food processing scores.

20. The system of claim 19, wherein the individual food processing score is a weight-based score or a calorie-based score.

21. The system of claim 19, wherein the processor is configured to determine the individual food processing score iFPS_{WF}^{j} for an individual j according to:
$${iFPS}_{W F}^{j} = {\sum }_{k}^{{D}_{j}} \frac{{w}_{k}^{j}}{{W}^{j}} {FPS}_{k}$$
where Dⱼ is a number of dishes consumed by the individual, W^{j} is a daily total amount of consumed food by the individual by weight, and wₖ^{j} is an amount consumed for each food item by weight.

22. The system of claim 19, wherein the processor is configured to determine the individual food processing score iFPS_{WC}^{j} for an individual j according to:
$${iFPS}_{W C}^{j} = {\sum }_{k}^{{D}_{j}} \frac{{c}_{k}^{j}}{{C}^{j}} {FPS}_{k}$$
where Dⱼ is a number of dishes consumed by the individual, C^{j} is a daily total amount of consumed food by the individual by calories, and cₖ^{j} is an amount consumed for each food item by calories.

23. A computer-implemented method of providing a precision nutrition prescription for an individual, the method comprising:
receiving an input comprising an identification of a food for consumption by an individual;
generating a vector of probabilities based on a nutrient profile for the food, the nutrient profile including nutrient content data for the food, and each probability of the vector representing a probability associated with a processing category for the food;
determining a food processing score based on the vector of probabilities;
generating a prescription for the individual based on the determined food processing score, the prescription comprising a recommendation for consumption of the food by the individual, a recommendation for consumption of an alternative food by the individual, or a combination thereof; and
outputting for display the determined prescription.

24. The computer-implemented method of claim 23, further comprising receiving an input including biological data of the individual, wherein generating a prescription for the individual is further based on the received biological data.

25. A precision nutrition engine, comprising:
a data source comprising a nutrient profile for each of a plurality of foods, the nutrient profile including nutrient content data for a food; and
a processor communicatively coupled to the data source and configured to:
receive an input comprising an identification of a food for consumption by an individual,
generate a vector of probabilities based on the nutrient profile for the food,
determine a food processing score based on the vector of probabilities, generate a prescription for the individual based on the determined food processing score, the prescription comprising a recommendation for consumption of the food by the individual, a recommendation for consumption of an alternative food by the individual, or a combination thereof, and
output for display the determined prescription.

26. The precision nutrition engine of claim 25, further comprising a data source including biological data of the individual, wherein the processor is further configured to receive the biological data and generate the prescription for the individual further based on the biological data.

27. A computer-implemented method of providing a precision nutrition prescription for an individual, the method comprising:
receiving an input comprising an identification of one or more foods consumed by an individual;
generating a vector of probabilities based on a nutrient profile for each of the one or more foods, the nutrient profile including nutrient content data for a consumed food, and each probability of the vector representing a probability associated with a processing category for the consumed food;
determining a food processing score based on the vector of probabilities for each of the one or more foods;
determine an individual food processing score based on the determined food processing scores;
generating a prescription for the individual based on the determined individual food processing score, the prescription comprising a recommendation of foods for consumption by the individual; and
outputting for display the determined prescription.

28. The computer-implemented method of claim 27, further comprising receiving an input including biological data of the individual, wherein generating a prescription for the individual is further based on the received biological data.

29. (canceled)

30. (canceled)
